(19) United States
(12) Patent Application Publication
(10) Pub. No.: US 2026/0286338 A1
(43) Pub. Date: Sep. 24, 2026

(54) USE OF IMAC TO IMPROVE RNA PURITY

(71) Applicant: ModernaTX, Inc., Cambridge, MA (US)

(72) Inventor: Matthew Burak, Cambridge, MA (US)

(73) Assignee: ModernaTX, Inc., Cambridge, MA (US)

(21) Appl. No.: 18/998,082

(22) PCT Filed: Jul. 24, 2023
(86) PCT No.: PCT/US2023/028435

§ 371 Date: Jan. 23, 2025

Related U.S. Application Data
(60) Provisional application No. 63/391,974, filed on Jul. 25, 2022.

(51) Int. Cl. C12N 15/10

(52) U.S. Cl. CPC C12N 15/101

(57) ABSTRACT

Described are methods and apparatuses for isolating and purifying mRNA using Immobilized Metal Affinity Chromatography (IMAC). The methods and apparatuses use IMAC matrices and IMAC conditions designed to selectively bind and elute mRNA, and are useful, for example, for isolating and purifying mRNA from in vitro-transcribed compositions. The methods and apparatuses also are useful for isolating and purifying linear or circular RNA having a poly(A) tail.

### CROSS-REFERENCE TO RELATED APPLICATIONS

This application claims priority to U.S. provisional application 63/391,974, filed Jul. 25, 2022, the entire contents of which are incorporated herein by reference.

### FIELD

Described are methods and apparatuses for isolating and purifying linear or circular polynucleotides comprising a poly(A) sequence, such as RNA comprising a poly(A) sequence (including mRNA), using Immobilized Metal Affinity Chromatography (IMAC).

### BACKGROUND

Immobilized Metal Affinity Chromatography (IMAC) has been used to purify proteins based on the affinity of their surface-exposed amino acids (especially histidine residues) for chelated metal ions. IMAC has found widespread application in the purification of recombinant histidine-tagged and pharmaceutical proteins, typically using Cu(II) and Ni(II) ions chelated by iminodiacetic acid (IDA) and nitrilotriacetic acid (NTA) ligands. Metal chelate ligands have been used as affinity agents in chromatography, and also have been immobilized on foams, membranes, biosensor chips, and in electrophoresis gels, and have been used as affinity precipitation agents.

WO 2002/046398 proposed the use of IMAC to separate double-stranded nucleic acid polymers from single-stranded nucleic acid polymers or to remove nucleotides and primers from PCR reactions. In particular, WO 2002/046398 reports that compounds containing a non-shielded purine or pyrimidine moiety (such as single-stranded nucleic acid molecules) exhibit affinity to an IMAC matrix while compounds that do not contain a non-shielded purine or pyrimidine moiety (such as double-stranded nucleic acid molecules) do not. Although WO 2002/046398 proposed that its approach could be used for mRNA, it does not describe how to implement IMAC to purify mRNA, let alone how to implement IMAC to purify mRNA from a composition comprising other substances that also could be bound by an IMAC ligand, such as histidine-tagged protein and tailless RNA.

Thus, there is a need for methods of isolating and purifying linear or circular polynucleotides comprising a poly(A) sequence, such as RNA comprising a poly(A) sequence (including mRNA), including a need for methods using IMAC to isolate and purify mRNA.

### SUMMARY

In accordance with some aspects, there are provided methods of purifying a polynucleotide having a poly(A) sequence, comprising contacting a composition comprising a polynucleotide having a poly(A) sequence with an immobilized metal affinity chromatography (IMAC) ligand, wherein the IMAC ligand binds the polynucleotide having a poly(A) sequence to form an IMAC-polynucleotide complex; separating the composition from the IMAC-polynucleotide complex; and recovering purified polynucleotide from the IMAC-polynucleotide complex, wherein the polynucleotide comprises a poly(A) sequence having a length of about 20 adenine nucleotides or longer.

In some aspects, the polynucleotide is a linear polynucleotide. In some aspects, the polynucleotide is a circular polynucleotide. In some aspects, the polynucleotide is an mRNA. In some aspects, the poly(A) sequence is a poly(A) tail of mRNA.

In some aspects, the IMAC ligand comprises an amine-carboxylic acid chelating group and a metal ion, such as nitrilotriacetic acid (—CH(COOH)N(CH2COOH)₂) (NTA) or iminodiacetic acid (—N(CH₂COOH)₂ (IDA). In some aspects, the IMAC ligand comprises a metal ion selected from Cu²⁺, Zn²⁺, Ni²⁺, and Co²⁺.

In some aspects, the IMAC ligand is immobilized on a substrate. In some aspects, the substrate comprises an IMAC matrix comprising a polymer having the IMAC ligand attached thereto. In some aspects, the IMAC matrix is a chromatography resin.

In some aspects, the contacting comprises passing the composition through a chromatography column containing the IMAC ligand.

In some aspects, the composition contacted with the IMAC ligand has a pH<7 and >5. In some aspects, the composition contacted with the IMAC ligand has a pH of about 6. In some aspects, the composition contacted with the IMAC ligand comprises NaCl at a concentration of from about 100 mM to about 250 mM. In some aspects, the composition contacted with the IMAC ligand comprises ethanol at a concentration of about 10% v/v. In some aspects, the composition contacted with the IMAC ligand does not include ethanol.

In some aspects, recovering purified polynucleotide from the IMAC-polynucleotide complex is effected using an elution buffer. In some aspects, the elution buffer comprises imidazole, has a pH of about 8, and comprises NaCl at a concentration of about 200 nM.

In some aspects, the polynucleotide has a length of from about 50 to about 5000 nucleotides. In some aspects, the polynucleotide has a length of from about 3000 to about 5000 nucleotides. In some aspects, the poly(A) sequence has a length of from about 50 to about 300 adenine nucleotides.

In some aspects, the composition comprises a histidine-tagged protein, and the method is effective to separate and/or purify the polynucleotide from the histidine-tagged protein. In some aspects, the composition comprises tailless RNA, and the method is effective to separate and/or purify the polynucleotide from the tailless RNA.

In some aspects, the polynucleotide is an mRNA and the composition is an in vitro transcribed-(IVT) mRNA comprising the mRNA and histidine-tagged T7 RNA polymerase, wherein the method is effective to separate and/or purify the mRNA from the T7 RNA polymerase. In some aspects, the polynucleotide is an mRNA and the composition is an in vitro transcribed-(IVT) mRNA composition comprising the mRNA and linearized DNA plasmid, histidine-tagged T7 RNA polymerase, and nucleotide triphosphates.

In some aspects, the composition comprises a chelating agent, and the method further comprises, prior to contacting the composition with the IMAC ligand, conducting a buffer exchange to remove chelating agent from the composition.

In some aspects, a method as described herein further comprises a heat denaturation step.

In some aspects, the composition does not contain DNA.

Also provided are immobilized metal affinity chromatography (IMAC) columns comprising an IMAC-polynucleotide complex comprising an IMAC ligand bound to a polynucleotide having a poly(A) sequence, wherein the poly(A) sequence has a length of about 20 adenine nucleotides or longer.

### BRIEF DESCRIPTION OF THE DRAWINGS

FIG. 1A illustrates binding of a nitrilotriacetic acid (—CH(COOH)N(CH2COOH)₂) (NTA) chelating group to a metal ion (e.g., Cu²⁺, Ni²⁻, Co²⁺ or Zn²⁺). As illustrated, NTA is a tetravalent metal binder.

FIG. 1B illustrates binding of an iminodiacetic acid (—N(CH2COOH)₂ (IDA) chelating group to a metal ion (e.g., Cu²⁺, Ni²⁺, Co²⁺ or Zn²⁺). As illustrated, IDA is a trivalent metal binder.

FIG. 2 presents static binding capacity curves (bound RNA (g/L) v. free RNA (g/L) for different IMAC resins assessed in Example 1.

FIG. 3 shows a comparison of the selectivity of RNA binding using Nuvia IMAC resin (top) or Fractogel EMD Chelate resin (bottom).

FIG. 4 shows a comparison of the selectivity of mRNA binding using a Nuvia IMAC resin, Fractogel EMD Chelate resin, or Oligo dT150 chromatography, with load samples containing 64±1% poly(A) tailed mRNA, and indicates that the Nuvia IMAC resin is capable of selectively binding poly(A) tailed mRNA in a similar manner to Oligo dT150, and achieves a tail purity of about 96%.

FIG. 5 shows mRNA binding to Nuvia IMAC resin at pH 6-8 (static binding of sample with >95% tail purity).

FIG. 6 shows mRNA binding to Nuvia IMAC resin at pH 6-8 (column binding of load sample with ~70% tail purity).

FIG. 7 shows mRNA binding to Nuvia IMAC resin at 0-500 mM NaCl (static binding of sample with >95% tail purity).

FIG. 8 shows mRNA binding to Nuvia IMAC resin at 0-500 mM NaCl (column binding of load sample with ~70% tail purity).

FIG. 9 shows mRNA binding to Nuvia IMAC resin at 0-20% v/v ethanol (static binding of sample with >95% tail purity).

FIG. 10 shows mRNA binding to Nuvia IMAC resin at 0-20% v/v ethanol (column binding of load sample with ~70% tail purity).

### DETAILED DESCRIPTION

### Definitions

The term “about” as used herein in connection with a numerical value denotes a degree of approximation, as would be understood to a person skilled in the art. Thus, the term “about” means that the numerical value so modified is not limited to the exact number set forth, but can vary to some extent based on the context in which it is used as would be understood by a person skilled in the art. Unless otherwise apparent from the context or convention in the art, “about” means up to plus or minus 10% of the particular term. The term “approximately” as used herein in connection with a numerical value means up to +/−5% of the number set forth.

As used herein with respect to two or more moieties or physical materials, the term “associated with” means that the moieties are connected with or interact with one another, either directly or indirectly. For instance, two moieties can be directly connected (e.g., covalently, ionically, or via other molecular interactions) or connected via one or more additional moieties that serves as a linking agent, to form a structure that is sufficiently stable so that the moieties remain physically associated under the conditions in which the structure is used (e.g., physiological conditions). In some embodiments, two or more moieties or physical materials are associated with each other when they are conjugated, linked, attached, or tethered to each other.

As used herein, the term “comparing” or “compared to” refers to the identification of the similarity or dissimiliarty of a particular property or measurable characteristic (e.g., amount) in one item relative to the same property or measurable characteristic (e.g., amount) in another item. For instance, a comparison can be a mathematical comparison of two or more values, e.g., of the levels of the biomarker(s) present in a ample. A comparison can be based on individual values, mean values, or average values. Comparing or comparison to can be in the context, for example, of comparing to a reference value, e.g., as compared to a reference blood plasma, serum, red blood cells (RBC) and/or tissue (e.g., liver, kidney, heart) biomarker level, and/or a reference serum, blood plasma, tissue (e.g., liver, kidney, or heart), and/or urinary biomarker level, in a subject prior to treatment (e.g., prior to administration of a therapeutic agent) or in a normal or healthy subject.

As used herein, the term “contacting” means establishing physical contact between two or more substances. Unless otherwise specified or dictated by the context in which it is used, “contacting” includes contacting that occurs in vivo, in vitro, or ex vivo.

As used herein, the term “isolated” refers to a substance that has been separated from at least some of the components with which it was associated (whether in nature or in an experimental setting). Isolated substances may have varying levels of purity in reference to the substances from which they were isolated. Isolated substances may be separated from at least about 10%, about 20%, about 30%, about 40%, about 50%, about 60%, about 70%, about 80%, about 90%, or more of the other components from which they were isolated. In some contexts, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. By “substantially isolated” is meant that the substance is substantially separated from the environment in which it was formed or detected. Partial separation can include, for example, a composition enriched in the substance of interest.

As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a poly(A) sequence, and/or a polyadenylation signal. An mRNA may have a nucleotide sequence encoding one or more polypeptide(s). Translation of an mRNA, for example, in vivo translation of an mRNA inside a mammalian cell, may produce a polypeptide, including a protein (e.g., an antigen such as a vaccine antigen, or a therapeutic protein, or a diagnostic protein). Typically, the basic components of an mRNA molecule include at least a coding region, a 5′-untranslated region (5′UTR), a 3′UTR, a 5′ cap and a poly(A) sequence.

A UTR can be homologous or heterologous to the coding region in a polynucleotide. In some embodiments, the UTR is homologous to an ORF encoding one or more proteins or peptide epitopes. In some embodiments, the UTR is heterologous to an ORF encoding one or more proteins or peptide epitopes. In some embodiments, a polynucleotide comprises two or more 5′ UTRs or functional fragments thereof, each of which have the same or different nucleotide sequences. In some embodiments, a polynucleotide comprises two or more 3′ UTRs or functional fragments thereof, each of which have the same or different nucleotide sequences.

In some embodiments, a 5′ UTR or functional fragment thereof, 3′ UTR or functional fragment thereof, or any combination thereof is sequence optimized.

In some embodiments, a 5′ UTR or functional fragment thereof, 3′ UTR or functional fragment thereof, or any combination thereof, comprises at least one chemically modified nucleobase, e.g., 5-methoxyuracil.

In some embodiments, the 5′ UTR and the 3′ UTR are heterologous. In some embodiments, the 5′ UTR is derived from a different species than the 3′ UTR. In some embodiments, the 3′ UTR is derived from a different species than the 5′ UTR.

International Patent Application No. PCT/US2014/021522 (Publ. No. WO/2014/164253) provides a listing of exemplary UTRs that may be utilized in mRNAs as flanking regions to an ORF. This publication is incorporated by reference herein for this purpose.

Additional exemplary UTRs that may be utilized in polynucleotides discussed herein include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H-ATP synthase); a growth hormone (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

A 5′ UTR may be selected from the group consisting of a β-globin 5′ UTR; a 5′ UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

A 3′ UTR may be selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

A polynucleotide may comprise multiple UTRs, e.g., a double, a triple or a quadruple 5′ UTR or 3′ UTR. As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure,” “primary structure,” “secondary structure,” and “tertiary structure,” based on increasing organizational complexity.

As used herein, the term “nucleic acid” encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines (e.g., that confer improved properties such as binding affinity, nuclease resistance, chemical stability to a nucleic acid or a portion or segment thereof). Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof (e.g., that confers improved chemical and/or functional properties such as binding affinity, nuclease resistance, chemical stability to a nucleic acid or a portion or segment thereof).

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified (for example by alkylation, and/or by capping) and unmodified forms of the polynucleotide. More particularly, the term “polynucleotide” includes polydeoxyribonucleotides (containing 2-deoxy-D-ribose); polyribonucleotides (containing D-ribose), including tRNA, IRNA, hRNA, siRNA and mRNA, whether spliced or unspliced; any other type of polynucleotide that is an N- or C-glycoside of a purine or pyrimidine base; and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration that allows for base pairing and base stacking, such as is found in DNA and RNA. In some aspects, the polynucleotide comprises an mRNA. In some aspects, the mRNA is a synthetic mRNA. In some aspects, the synthetic mRNA comprises at least one non-natural nucleobase. In some aspects, the mRNA comprises a non-natural components at a position other than the nucleobase. In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

Examples of naturally-occurring nucleotides used for the production of RNA, e.g., in an IVT reaction as discussed herein, include adenosine triphosphate (ATP), guanosine triphosphate (GTP), cytidine triphosphate (CTP), uridine triphosphate (UTP), and 5-methyluridine triphosphate (m5UTP). For example, adenosine diphosphate (ADP), guanosine diphosphate (GDP), cytidine diphosphate (CDP), and/or uridine diphosphate (UDP) may be used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (e.g., vaccinia or ligase), a nucleotide labeled with a functional group to facilitate ligation/conjugation of cap or 5′ moiety (e.g., IRES), a nucleotide labeled with a 5′ PO4 to facilitate ligation of cap or 5′ moiety, or a nucleotide labeled with a functional group/protecting group that can be chemically or enzymatically cleaved.

Modified nucleotides may include modified nucleobases. For example, a polynucleotide (e.g., mRNA) as discussed herein may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U), and 2′-O-methyl uridine. For example a polynucleotide (e.g., mRNA) as discussed herein may include one or more (e.g., a combination of at least two (e.g., 2, 3, 4 or more)) of the foregoing modified nucleobases.

As used herein, pseudouridine (ψ) refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m1ψ) (also known as N1-methyl-pseudouridine), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, 1-methyl-3-(3-amino-3-carboxypropyl) pseudouridine (acp3 ψ), and 2′-O-methyl-pseudouridine (ψm).

As used herein, the term “substantially” refers to the qualitative condition of exhibiting total or near-total extent or degree of a characteristic or property. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “transcription” refers to methods to produce mRNA (e.g., an mRNA sequence or template) from DNA (e.g., a DNA template or sequence).

The term “IMAC matrix” as used herein means a medium that includes immobilized divalent metal ions capable of binding one or more nucleosides that are capable of binding divalent metal ions (e.g., adenine, guanine, cytosine, and uracil). An IMAC matrix generally comprises a polymer having a ligand attached thereto, where the ligand is capable of immobilizing (e.g., chelating) a divalent metal ion. Typical metal ions used for this purpose include Cu(II), Ni(II), Zn(II), and Co(II), which may be chelated by iminodiacetic acid (IDA) or nitrilotriacetic acid (NTA) ligands.

The term “ligand” as used herein generally refers to a molecule capable of binding a metal ion. As used herein, ligands are generally chemically bonded to a substrate, e.g., the IMAC matrix. As used herein, “IMAC ligand” refers to a ligand bound to a divalent metal ion. Thus, an IMAC matrix generally comprises a plurality of IMAC ligands.

The term “binding” as used herein with reference to binding to a IMAC ligand (e.g., an IMAC ligand comprising immobilized metal ions) means any chemical and/or physical interacting with the metal ions, including, without limitation, any one or more of hydrogen bonding, coordinate bonding, apolar bonding, ionic bonding, covalent bonding, electrostatic interaction, ionic interaction, and combinations of any thereof.

The terms “poly(A) sequence” and “poly(A) tail” as used herein refer to a chain of adenine nucleotides. In the context of mRNA, a “poly(A) tail” is a region of mRNA that is downstream, e.g., directly downstream (i.e., 3′), from the open reading frame and/or the 3′ UTR that contains multiple, consecutive adenosine monophosphates. (An open reading frame (ORF) is a continuous stretch of deoxyribonucleic acid (DNA) or RNA beginning with a start codon (e.g., methionine (ATG or AUG)) and ending with a stop codon (e.g., TAA, TAG or TGA, or UAA, UAG or UGA). An ORF typically encodes a protein.) A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates (adenine nucleotides). In some embodiments, a poly(A) tail contains 50 to 250 adenosine monophosphates (adenine nucleotides). In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

Where the following discussion describes aspects of the invention in the context of specific embodiments, it will be understood that the invention is not limited to those embodiments.

As noted above, described herein are methods and apparatuses for purifying linear or circular polynucleotides comprising a poly(A) sequence, such as RNA comprising a poly(A) sequence (including mRNA) using IMAC. The methods and apparatuses are useful for purifying such polynucleotides from compositions comprising the polynucleotides and other substances, including other substances that may be bound by IMAC ligands, such as histidine-tagged proteins (“His-tagged proteins”) and tailless RNA. For example, the methods and apparatuses are useful for purifying, e.g., mRNA, from compositions comprising tailless RNA. The methods and apparatuses also are useful for purifying, e.g., mRNA, from in vitro transcribed-(IVT) compositions which may include other substances that may be bound by IMAC ligands, such as His-tagged T7 RNA polymerase and tailless RNA. The methods and apparatuses described herein are useful for separation, isolation, purification, quantitation, etc. While primarily described with reference to mRNA, the methods and apparatuses described herein are useful for isolating and/or purifying any linear or circular polynucleotide comprising a poly(A) sequence, as discussed in more detail below.

As noted above, an IMAC ligand will bind to, e.g., adenine residues of a poly(A) tail of a polynucleotide molecule, such as a poly(A) tail of an mRNA molecule. However, sometimes there is a need to purify a linear or circular polynucleotide comprising a poly(A) sequence from a compositions that includes other substances that also could be bound by IMAC ligands, such as histidine-tagged proteins (e.g., histidine-tagged T7 RNA polymerase) and tailless RNA. In order to be able to use IMAC to isolate, separate, and/or purify linear or circular polynucleotide comprising a poly(A) sequence from such compositions, IMAC techniques had to be developed that would enhance selectivity for linear or circular polynucleotide comprising a poly(A) sequence relative to other substances that may be present. For example, in order to be able to use IMAC to isolate, separate, and/or purify mRNA from IVT compositions, IMAC techniques had to be developed that would enhance selectivity for mRNA relative to other substances that may be present that also could be bound by IMAC ligands, such as histidine-tagged proteins (e.g., histidine-tagged T7 RNA polymerase) and tailless RNA.

The present inventors surprisingly determined that by using specific types of IMAC resins and specific IMAC conditions, IMAC can be used to selectively and effectively isolate, separate, and/or purify linear or circular polynucleotide comprising a poly(A) sequence from a composition comprising other substances that may be bound by IMAC ligands. For example, the present inventors surprisingly determined that by using specific types of IMAC resins and specific IMAC conditions, IMAC can be used to selectively and effectively isolate, separate, and/or purify mRNA from IVT compositions with a high degree of efficiency.

Thus, provided herein are methods of purifying linear or circular polynucleotides comprising a poly(A) sequence, such as mRNA having a poly(A) tail (referred to generally as “target polynucleotide”), comprising contacting a composition comprising target polynucleotide with an immobilized metal affinity chromatography (IMAC) ligand as described herein, wherein the ligand binds the target polynucleotide to form an IMAC-target polynucleotide complex; separating the composition from the IMAC-target polynucleotide complex; and recovering purified target polynucleotide from the IMAC-target polynucleotide complex. In some embodiments, the methods comprise contacting a composition comprising mRNA having a poly(A) tail with an IMAC ligand as described herein, wherein the ligand binds the mRNA to form an IMAC-mRNA complex; separating the composition from the IMAC-mRNA complex; and recovering purified mRNA from the IMAC-mRNA complex.

Also provided herein are apparatuses comprising, e.g., an IMAC ligand immobilized on a solid support, wherein the ligand is bound to target polynucleotide, forming an IMAC-target polynucleotide complex. Also provided herein are apparatuses comprising, e.g., an IMAC ligand immobilized on a solid support, wherein the ligand binds mRNA having a poly(A) tail to form an IMAC-mRNA complex.

### Target Polynucleotide

In some aspects the target polynucleotide (e.g., the polynucleotide being separated or purified from a composition) is a linear or circular polynucleotide comprising a poly(A) sequence. The poly(A) sequence may be of any length sufficient to bind to an IMAC ligand as described herein. For example, in some aspects, the poly(A) sequence has a length of about 20 adenine nucleotides or longer, including a length of 18, 19, 20, or more, adenine nucleotides. In some aspects, the poly(A) sequence has a length of about 20 adenine nucleotides. In some aspects, the poly(A) sequence has a length of about 20 adenine nucleotides or longer. In some aspects, the poly(A) sequence has a length of from 50 to 300 adenine nucleotides. In some aspects, the poly(A) sequence has a length of about 50-250 adenine nucleotides. In any aspects, the poly(A) sequence may be a poly(A) tail, e.g., may be present at the 5′ end of the polynucleotide.

The total length of the target polynucleotide is not particularly limited. In some aspects, the target polynucleotide has a length of from about 50 to about 5000 nucleotides, including from 50 to 5000 nucleotides, including from about 500 to about 5000 nucleotides, including from 500 to 5000 nucleotides. In some aspects, the target polynucleotide has a length of from about 3000 to about 5000 nucleotides, including from 3000 to 5000 nucleotides. In some aspects, the target polynucleotide has a length of from about 2000 to about 3000 nucleotides, including from 2000 to 3000 nucleotides.

Other than the presence of a poly(A) sequence, the identity of the target polynucleotide (e.g., the polynucleotide being separated or purified from a composition) is not particularly limited, and generally includes any nucleic acid polymer. The target polynucleotide may include a substitution and/or modification. In some embodiments, the substitution and/or modification is in one or more bases and/or sugars. For example, in some embodiments a target polynucleotide (e.g., mRNA) includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified polynucleotide (e.g., mRNA) includes a 2′-O-alkylated ribose group. In some embodiments, a modified polynucleotide (e.g., mRNA) includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a target polynucleotide (e.g., mRNA) is heterogeneous in backbone composition thereby containing any possible combination of polymer units linked together.

In some aspects, the target polynucleotide is target mRNA (e.g., mRNA being separated or purified from a composition). In some aspects the target mRNA has a length of from about 50 to about 5000 nucleotides, including from 50 to 5000 nucleotides. In some aspects, the target mRNA has a length of from 4000 to 5000 nucleotides. In some aspects, the target mRNA has a length from about 500 to about 5000 nucleotides, including from 500 to 5000 nucleotides. In some aspects, the target polynucleotide has a length of from about 3000 to about 5000 nucleotides, including from 3000 to 5000 nucleotides. In some aspects, the target polynucleotide has a length of from about 2000 to about 3000 nucleotides, including from 2000 to 3000 nucleotides. As noted above, the target mRNA typically has a poly(A) tail, such as a poly(A) tail of from 50 to 300 adenine nucleotides in length, or of from 50-250 adenine nucleotides in length.

The target mRNA may be present in an in vitro transcribed-(IVT) composition. That is, the target mRNA may be prepared by a method comprising in vitro transcription, and present in the resulting IVT composition. An IVT composition typically includes linearized DNA plasmid, histidine-tagged T7 RNA polymerase, nucleotide triphosphates, and other components. For example, an IVT composition may include linearized DNA plasmid, histidine-tagged T7 RNA polymerase, pyrophosphatase, DNase I, nucleotide triphosphates, and buffer (e.g., tris(hydroxymethyl)aminomethane (Tris), HCl, magnesium acetate (MgOAc), dithiothreitol (DTT), spermidine). An IVT composition also may include tailless RNA. If the IVT composition includes ethylenediaminetetraacetic acid (EDTA) (or a similar chelating agent), a buffer exchange can be performed to remove the EDTA (or similar chelating agent) prior to carrying out a method as described herein.

As noted above, while primarily described with reference to mRNA, the methods and apparatuses described herein also are useful for isolating and/or purifying other linear or circular polynucleotides comprising a poly(A) sequence. Thus, in some aspects, the methods and apparatuses described herein are used for isolating and/or purifying a linear polynucleotide comprising a poly(A) sequence that is not an mRNA, such as RNA that is not mRNA, or another linear polynucleotide comprising a poly(A) sequence. In other aspects, the methods and apparatuses described herein are used for isolating and/or purifying a circular polynucleotide comprising a poly(A) sequence that is not an mRNA, such as circular RNA that is not mRNA, or another circular polynucleotide comprising a poly(A) sequence.

Throughout the following description, it should be understood that any circular or linear polynucleotide having a poly(A) sequence can be substituted for the target mRNA in the methods and apparatuses described, i.e., that the target for isolation, purification, etc., can be any circular or linear polynucleotide having a poly(A) sequence as disclosed herein (e.g., a poly(A) sequence having a length of about 20 adenine nucleotides or longer).

### IMAC Ligands

The present inventors surprisingly determined that specific types of IMAC ligands are particularly useful for isolating and purifying polynucleotides having a poly(A) sequence as disclosed herein (e.g., a poly(A) sequence having a length of about 20 adenine nucleotides or longer), such as mRNA, and for selectively and effectively isolating and purifying mRNA from IVT compositions.

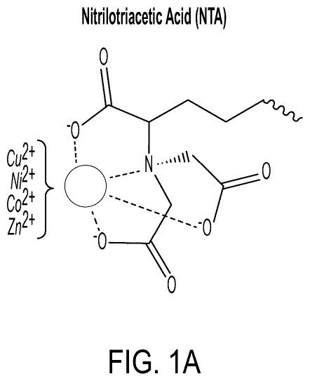

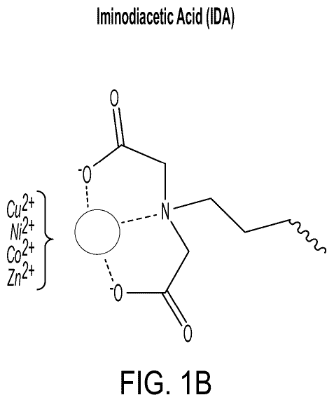

In some aspects, the IMAC ligand comprises an amine-carboxylic acid chelating group and a metal ion. In some aspects, the IMAC ligand comprises an amine-carboxylic acid chelating group selected from nitrilotriacetic acid (—CH(COOH)N(CH2COOH)₂) (NTA) and iminodiacetic acid (—N(CH₂COOH)₂ (IDA). As illustrated in FIG. 1A and FIG. 1B, NTA is a tetravalent metal binder while IDA is a trivalent metal binder.

In some aspects the IMAC ligand comprises a divalent metal ion. In some aspects the IMAC ligand comprises a divalent metal ion selected from Cu²⁺, Ni²⁺, Co²⁺ or Zn²⁺.

The IMAC ligand typically is immobilized on or associated with a substrate, in a form referred to herein as an “IMAC matrix.” As noted above, an IMAC matrix generally comprises a polymer having an IMAC ligand attached thereto.

IMAC matrices comprising a metal ion can be prepared by methodologies known in the field, such as by using a metal salt that is soluble in a metal charging buffer having a counterion that does not adversely affect the IMAC ligand or the substrate to which the ligand is bound. Typical metal salts for this purpose include metal halides such as metal fluorides, metal chlorides, metal bromides, metal iodides, and mixture thereof; metal carboxylates, metal carbonates or bicarbonates, metal nitrates, metal phosphates, metal sulfates, metal oxychlorides, and similar metal complexes. Typically, metal chlorides are used.

Suitable polymer substrates for IMAC ligand functionalization are known in the field, and include sepharose, chemically- and/or physically-modified sepharose, agarose, chemically- and/or physically-modified agarose, other polymeric sugars or chemically- and/or physically-modified versions thereof, cellulose, chemically- and/or physically-modified cellulose, polyolefins, chemically- and/or physically-modified polyolefins, polydienes, chemically- and/or physically-modified polydienes, polyurethanes, chemically- and/or physically-modified polyurethanes, polypeptides, chemically- and/or physically-modified polypeptides, polyamides, chemically- and/or physically-modified polyimides, polyalkyleneoxides, chemically- and/or physically-modified polyalkyleneoxides (such as polyethyleneglycols), chemically- and/or physically-modified polyethyleneglycols, silicones, elastomers, thermoplastics, thermoplastic elastomers, or any other polymeric substrate.

Suitable supports for IMAC matrices are known in the field, and include non-capillary columns, capillary columns, gels, chip surfaces, microplate surfaces, porous foams, porous resin, polymer beads (including macroreticular beads), surfaces of non-porous monolithic structures such as inorganic monolithic structures used in catalytic converters or polymeric structures such as epoxide resins including CIM monoliths made by BIA Separations of Ljubljana, Slovenia, or the like. Suitable supports also include membranes, such as impermeable membranes, permeable membranes, semi-permeable membranes, macroporous fibrous membranes, and chemically- and/or physically-modified membranes. Suitable supports also include inorganic supports, including silicas, silicates, aluminas, silca-aluminas, zeolites, mordenties, fugasites, aluminates, clays, monoliths, honeycombed monoliths, etc. Suitable supports also include metallic supports, including gold, gold alloys, platinum, platinum alloys, silver, silver alloys, iron, iron alloys (such as any steel), copper, copper alloys such as brass or bronze, tin and tin alloys, aluminum and aluminum alloys, silicon and silicon alloys, other semiconductors, etc.

Suitable chemical and physical modification processes for the preparation of IMAC matrices are known in the field, and include chemical functionalization with reactive chemical agents, ion and/or atom bombardment and/or implantation, reactive extrusion, chemical etching, chemical deposition, and other chemical and/or physical modifications that permit an IMAC ligand to be bounded to a substrate or support.

In general, the IMAC matrix is selected to have sufficiently large enough flow channels and/or pores to allow for diffusion of large polynucleotide molecules, such as target mRNA. The IMAC resin also may be selected to have a suitable linker length that does not result in unacceptable non-specific binding.

In some aspects, the IMAC matrix is a Nuvia IMAC resin. Nuvia IMAC resins have an NTA ligand, a metal ion capacity of about 18 μmol/mL, an average particle size of about 50 μm, and a pore size of about 90 nm.

In some aspects, the IMAC matrix is or is comprised in a chromatography resin. In some aspects, a chromatography resin comprising an IMAC matrix is provided in a chromatography column (referred to herein as an “IMAC column”). In accordance with such aspects, a method as described herein may comprise passing the composition through an IMAC column. In such methods, the IMAC matrix of the IMAC column will capture polynucleotide having a poly(A) tail (e.g., mRNA) via binding between the IMAC ligand and poly(A) tail.

In some aspects, the IMAC matrix is provided on a membrane, such as a membrane coated with or impregnated with an IMAC matrix. In accordance with such aspects, a method as described herein may comprise passing the composition through the membrane. In such methods, the membrane may act as a filter with the IMAC ligand binding to and retaining polynucleotide having a poly(A) tail (e.g., mRNA).

In some aspects, the IMAC matrix is provided on a magnetic object, such as a bead, stirring rod, or the like. For example, a magnetic object may be coated with or have an IMAC matrix bonded thereto and/or deposited thereon or in pores thereof. In accordance with such aspects, a method as described herein may comprise contacting the composition with the magnetic object. In such methods, the magnetic object may capture polynucleotide having a poly(A) tail (e.g., mRNA) via binding between the IMAC ligand and the polynucleotide having a poly(A) tail. Such methods may further comprise separating the magnetic object (with captured polynucleotide) from the composition.

In some aspects, the IMAC matrix is provided on a metallic object, such as a bead. For example, a metallic object may be coated with or have an IMAC matrix bonded thereto and/or deposited thereon or in pores thereof. In accordance with such aspects, a method as described herein may comprise contacting the composition with the metallic object. In such methods, the metallic object may capture polynucleotide having a poly(A) tail (e.g., mRNA) via binding between the IMAC ligand and the polynucleotide having a poly(A) tail. Such methods may further comprise separating the metallic object (with captured polynucleotide) from the composition, such as with magnets, filtration, etc.

In some aspects, an IMAC matrix is provided on the surface of a well of a microplate. In accordance with such aspects, a method as described herein may comprise contacting the composition with the microplate. In such methods, the microplate may capture polynucleotide having a poly(A) tail (e.g., mRNA) via binding between the IMAC ligand and the polynucleotide having a poly(A) tail. Such methods may further comprise removing the composition from the microplate (with captured polynucleotide), such as by washing.

In accordance with any of the foregoing aspects, the composition may be loaded onto the IMAC matrix at an appropriate load for the type of substrate being used. Load challenge (grams of polynucleotide per liter of resin) typically is dependent on the size of the polynucleotide and the specific IMAC matrix used. For example, with the Nuvia IMAC resin, a polynucleotide (e.g., an mRNA molecule) having a length about 4,800 nucleotides can be loaded at about 2 g/L.

In accordance with any of the foregoing aspects, the method may comprise incubating the composition on the IMAC matrix for a suitable period of time to permit binding of the target polynucleotide and, if applicable, flowthrough of the composition. Pore diffusion is time dependent, and a long incubation period (residence time), increases the likelihood of binding. Thus, a method as described herein may include an incubation period (residence time) of from 5 minutes to 120 minutes, including about 15 minutes, about 20 minutes, about 30 minutes, about 60 minutes, about 75 minutes, about 90 minutes, and about 105 minutes. In the examples below, a 20 minute residence time on the Nuvia IMAC columns was found to achieve suitable binding of target mRNA.

In accordance with any of the foregoing aspects, the target polynucleotide (e.g., mRNA) may be recovered from the IMAC matrix (e.g., eluted from an IMAC column or released and collected from another substrate) by any suitable method. Generally, elution is accomplished by adding an appropriate volume of an elution buffer to the IMAC matrix and collecting the eluant. Suitable elution buffers are discussed in more detail below.

### Chromatography Conditions

As noted above, an IMAC ligand as disclosed herein (e.g., comprising an NTA or IDA chelating group and a divalent metal ion) generally is capable of binding to, e.g., adenine residues of a poly(A) sequence of a polynucleotide molecule, such as the poly(A) tail of an mRNA molecule. However, a target polynucleotide may be present in a composition, such as an IVT composition, that includes other substances that also could be bound by IMAC ligands, such as histidine-tagged proteins (e.g., histidine-tagged T7 RNA polymerase) and tailless RNA. In order to be able to use IMAC to isolate, separate, and/or purify, e.g., mRNA, from IVT compositions, IMAC techniques had to be developed that would enhance selectivity for polynucleotide having a poly(A) tail (e.g., mRNA) relative to other substances that may be present in the composition that could be bound by IMAC ligands.

Prior studies have indicated that pH, salt concentration, and the presence of neutral solutes may impact RNA binding to IMAC ligands. See, e.g., Fu, et al. J Mol Recognit. July-August 2006; 19 (4): 348-53; Potty, et al. J Chromatogr A. 2006 May 19; 1115 (1-2): 88-92. However, the ability to develop IMAC conditions that would permit the use of IMAC to isolate and purify mRNA from an IVT composition have not heretofore been proposed.

As illustrated in the examples below, the present inventors determined specific IMAC conditions that can be used to increase selectivity for mRNA and permit the use of IMAC to isolate, separate, and/or purify mRNA from an IVT composition. For example, although RNA is capable of binding IMAC ligands at pH 6-7.5, the present inventors have determined that using a slightly acid pH (as discussed below) may help limit nonspecific protein binding. Further, the present inventors have determined that, e.g., ethanol, may enhance capacity at the expense of specificity for poly(A) tailed versus tailless RNA binding; thus, in embodiments where specificity is of primary import (e.g., in methods conducted to improve tail purity), ethanol may be absent from the composition applied to the IMAC resin.

In accordance with one aspect, the inventors determined that selectivity for polynucleotides having a poly(A) tail (e.g., mRNA) can be increased by using a slightly acid pH. In accordance with another aspect, the inventors determined that selectivity for polynucleotides having a poly(A) sequence (e.g., mRNA) can be increased by adjusting the ionic strength of the composition using a suitable salt (e.g., NaCl) at a certain threshold concentration (e.g., ≥100 mM). In accordance with another aspect, the inventors determined that binding of polynucleotides having a poly(A) sequence (e.g., mRNA) can be increased by the presence of an organic solute, such as by the additional of ethanol to the composition, although ethanol may reduce specificity for poly(A)-tailed versus tailless polynucleotide binding.

Thus, in some aspects, the composition subject to IMAC chromatography as described herein has a pH<7, including a pH of 6 to 6.5, such as a pH of about 6, including a pH of 6. As noted above, the present inventors have determined that using a slightly acid pH, such as a pH of about 6 (including a pH of 6) limit nonspecific protein binding. For example, histidine-tagged T7 polymerase binds to IMAC ligands (e.g., to Nuva IMAC resin) under slightly basic conditions, exhibiting weak binding at pH<6 and strong binding at pH >6. However, the use of a more acidic buffer (e.g., pH=5) decreases binding of both polynucleotide having a poly(A) sequence (e.g., mRNA) and histidine-tagged T7 polymerase, likely due to protonation of adenine and histidine moieties, respectively. For example, the adenosine NI group may become protonated and no longer be able to bind the metal of the IMAC ligand. Additionally, without being bound by theory, it is believed metal hydroxide complex formation may occur on basic conditions, resulting in a negative charge density that may interfere with binding of IMAC ligands to nucleobases. Thus, in aspects of the methods described herein, a pH >5 is used, such as a pH of about 6 to about 6.5, including a pH of about 6, such as a pH of 6.

If the pH of the composition is not at the desired pH, the pH can be adjusted, e.g., using an appropriate buffer before subjecting the composition to IMAC chromatography. Examples of suitable buffers include 2-(N-morpholino) ethanesulfonic acid buffers (MES) and 1,3-bis(tris(hydroxymethyl)methylamino) propane (Bis-tris propane).

Additionally or alternatively, in some aspects, the composition subject to IMAC chromatography as described herein has a selected ionic strength, such as provided by addition of a salt. Suitable salts for this purpose include NaCl and KCl. A suitable concentration of a salt such as NaCl or KCl is from about 100 nM to about 250 nM, including 100 nM to 250 nM. Without being bound by theory, it is believed salt counterions may provide electrostatic shielding and facilitate binding of IMAC ligands to nucleobases. On the other hand, a concentration of, e.g., NaCl as high as 500 nM may reduce specificity and, for example, promote binding of tailless RNA.

Additionally or alternatively, in some aspects, the composition subject to IMAC chromatography as described herein has an ethanol concentration of 10-20% v/v, such as an ethanol concentration of 10% v/v, 15% v/v, or 20% v/v, including an ethanol concentration of 10% v/v. Without being bound by theory, it is believed ethanol may strip away water coordinated with the IMAC ligand, thereby freeing up IMAC ligand for binding with target polynucleotide (e.g. mRNA), e.g., thereby enhancing nucleobase binding. A similar effect may be seen with other polar solvents, such as propanol, isopropanol, methanol, and DMSO. As noted above, in embodiments where tailed polynucleotide (e.g., tailed mRNA) is the target, such solvents (ethanol, propanol, isopropanol, methanol, DMSO, etc.) may be absent so as not to promote binding of tailless polynucleotide (e.g., tailless RNA).

In some aspects, where an objective is complete polynucleotide binding, the composition comprising target polynucleotide (e.g., target mRNA) is prepared with 50 mM MES buffer at pH 6.0, 250 mM NaCl, and 10% v/v ethanol prior to being applied to the IMAC matrix.

In some aspects, where an objective is tail purity (e.g., mRNA with high poly(A) tail purity), the composition comprising target polynucleotide (e.g., mRNA) is prepared with 50 mM MES buffer at pH 6.0, and 250 mM NaCl prior to being applied to the IMAC matrix.

As noted above, in any aspects, if the composition includes EDTA, a buffer exchange can be carried out to remove the EDTA prior to application to the IMAC matrix.

As noted above, after loading the composition onto the IMAC matrix, incubating for a suitable period of time, and collecting flowthrough, the target polynucleotide (e.g., mRNA) may be recovered from the IMAC matrix (e.g., eluted from an IMAC column or released and collected from another substrate) by any suitable method. Generally, elution is accomplished by adding an appropriate volume of an elution buffer to the IMAC matrix and collecting the eluant.

WO 2002/046398 disclosed the use of a buffer comprising imidazole to elute RNA from its IMAC columns. However, an imidazole elution buffer also elutes histidine-tagged proteins. In order to be able to isolate, separate, and/or purify, e.g., mRNA from, e.g., an IVT composition, selective elution of mRNA versus histidine-tagged protein would be advantageous. In that regard, the present inventors surprisingly determined that an elution buffer having a pH of from about 8 to about 9 (including a pH from 8 to 9) and a salt concentration of from 0 to about 200 mM NaCl of KCl can be used to selectively elute polynucleotide having a poly(A) tail (e.g., mRNA) while retaining His-tagged proteins (such as T7 RNA polymerase). For example, as illustrated in the example section below, an elution buffer comprising imidazole, 50 mM Tris at pH=8 and 200 mM NaCl is effective to selectively elute mRNA while retaining His-tagged proteins (such as T7 RNA polymerase). This has never been demonstrated before.

Once the target polynucleotide (e.g., mRNA) has been eluted, if desired, metal ions can be stripped from the IMAC matrix. For example, an EDTA solution can used be to strip metal ions from an IMAC matrix.

In accordance with any of the foregoing aspects, a method as described herein may further comprise a heat denaturation step. In some aspects, the methods described herein are used in conjunction with other separation techniques, including other chromatographic techniques. In such embodiments, a single chromatography column may be used that comprises an IMAC zone and a non-IMAC zone, where the non-IMAC zone is selected to separate other substances based on other properties. Additionally or alternatively an IMAC apparatus as described herein may be run in-line with other apparatus configured to conduct other separation techniques.

The following examples are provided for illustration, and are not limiting of the scope of the invention disclosed herein.

### EXAMPLES

### Example 1. Selection of IMAC Matrices

The ability of different IMAC resins to bind RNA was assessed in a static binding assay. The following IMAC resins were evaluated using Ni(II) as the metal ion:

| | | | | | Static |
|---|---|---|---|---|---|
| | | Metal Ion | Particle | Pore | Binding |
| | | Capacity | Size | Size | Capacity |
| Resin | Ligand | (μmol/mL) | (μm) | (nm) | (g/L) |
| Nuvia IMAC\| | NTA | 18 | 50 | 90 | 2.1 ± 0.1 |
| (BioRad) | | | | | |
| Fractogel | IDA | 75 | 75 | 80 | 1.4 ± 0.2 |
| EMD Chelate | | | | | |
| (Millipore) | | | | | |
| HisPur | NTA | — | — | — | 0.8 ± 0.2 |
| Ni-NTA | | | | | |
| (Thermo | | | | | |
| Fisher) | | | | | |
| Ni | NTA | 15 | 90 | 40 | 0.6 ± 0.1 |
| Sepharaose 6 | | | | | |
| Fast Flow | | | | | |
| (Takara Bio) | | | | | |
| HisPur | NTA | 15 | 100 | — | 0.6 ± 0.1 |
| Ni-NTA | | | | | |
| Superflow | | | | | |
| (Thermo | | | | | |
| Fisher) | | | | | |
| Ni Chelating | IDA | 30 | — | — | 0.6 ± 0.1 |
| Resin | | | | | |
| (Cytiva) | | | | | |

Microplates (50 μL) were loaded with resin and equilibrated with IMAC equilibration buffer (50 mM HEPES, pH=7; 250 mM NaCl). Waste was removed using a positive pressure manifold. Purified mRNA (~4,800 nt, >95% tail purity, >80% size purity) was formulated in the same buffer (50 mM HEPES, pH=7; 250 mM NaCl), added at a load of 2-10 g/L resin, and incubated for 60 minutes. Flowthrough was collected using a positive pressure manifold. An IMAC elution buffer (50 mM Tris, pH=8; 200 mM imidazole) was added, incubated for 30 minutes, and the eluate was collected using a positive pressure manifold. RNA present in the flowthrough and eluate was quantified (by measuring absorbance at 260 nm).

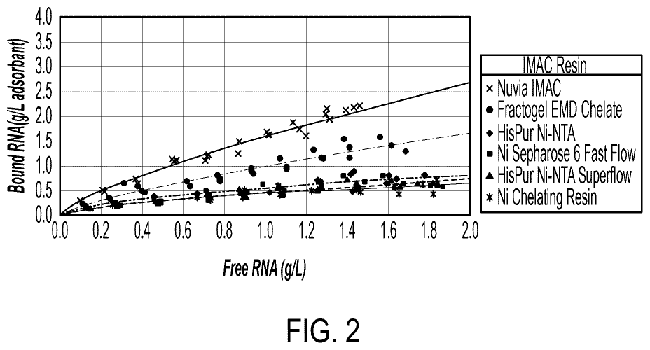

FIG. 2 presents the resulting static binding capacity curves (bound RNA (g/L) versus free RNA (g/L)). As seen in the figure, the Nuvia IMAC resin exhibited the greatest binding capacity, followed by the Fractogel EMD Chelate resin. These results indicate that resins with a larger pore size exhibited greater static binding capacity for RNA.

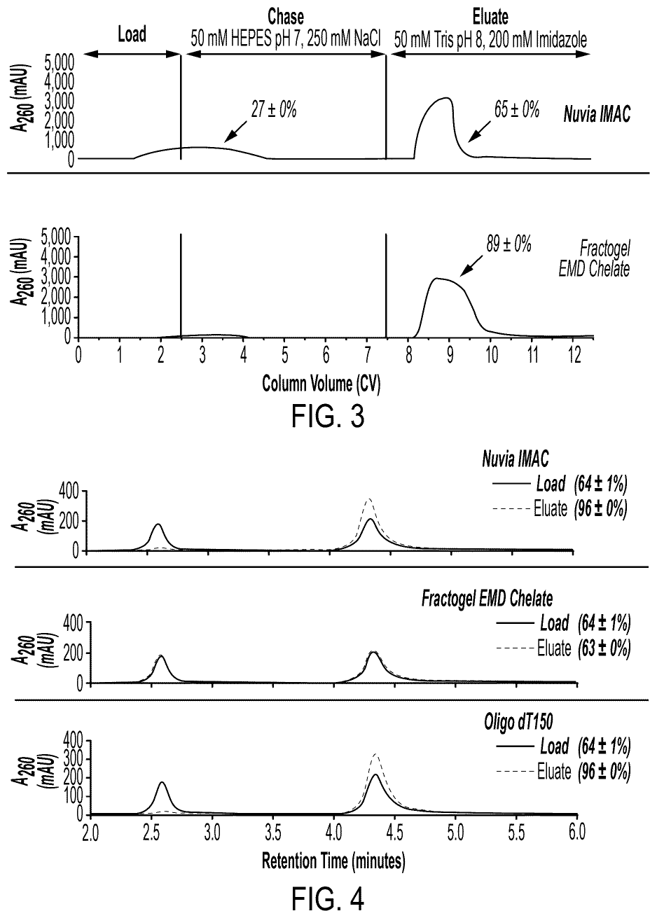

The Nuvia IMAC resin and Fractogel EMD Chelate resin were selected for further evaluation of their ability to selectively bind poly(A) RNA (e.g., mRNA). Columns (5 mL) were prepared and loaded with poly(A) RNA (~4,800 nt, ~70% tail purity) in buffer (50 mM HEPES, pH=7; 250 mM NaCl), with a retention time of 20 minutes, and flowthrough was collected. A chase was conducted using the same buffer (50 mM HEPES, pH=7; 250 mM NaCl) and retention time of 20 minutes, and flowthrough was collected. Elution was conducted using an IMAC elution buffer (50 mM Tris, pH=8; 200 mM imidazole) and a retention time of 5 minutes, and the eluate was collected. Poly(A) RNA present in the flowthrough and eluate was quantified (by measuring absorbance at 260 nm). Results are shown in FIG. 3.

The results show that the Fractogel EMD Chelate resin shows less selectivity, with less RNA in the flowthrough and chase samples as compared to the Nuvia IMAC resin.

The ability of the Nuvia IMAC resin and Fractogel EMD Chelate resin to selectively bind poly(A) RNA (e.g., mRNA) also was assessed, using an OligodT150 (150-mer deoxythymidine) resin (Thermo Fisher) as a comparator. FIG. 4 shows the tail purity of the load and eluate samples collected from the Nuvia IMAC, Fractogel EMD Chelate, and Oligo dT150 chromatography runs. Tail purity was assessed by HPLC reverse phase ion pair (TAE) analysis using a C18 column. The results show the Nuvia IMAC resin achieved a tail purity comparable to Oligo dT150.

### Example 2. Selection of IMAC Conditions for Selective Binding of Poly(A) RNA

The Nuvia IMAC resin was used in further experiments to identify IMAC conditions that would promote selective binding of poly(A) RNA, including pH, salt concentration, and ethanol, using mRNA samples with different tail purity as test materials.

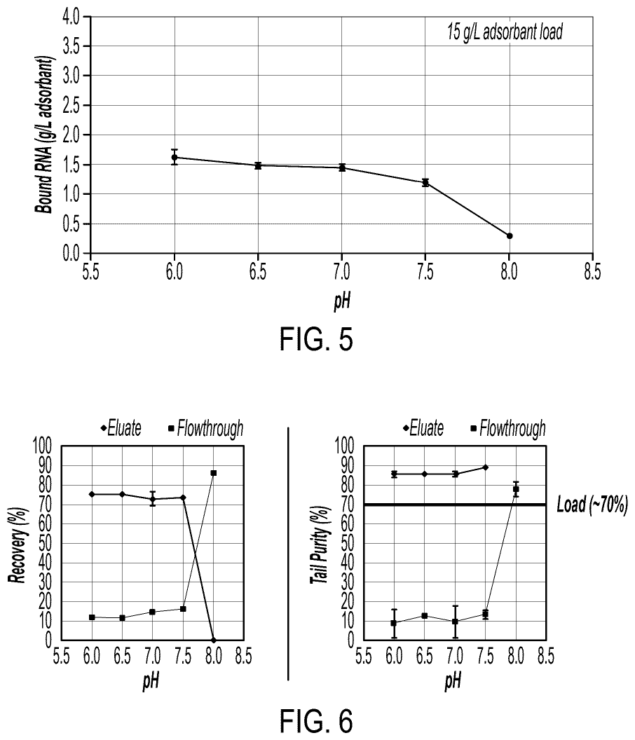

Microplates (50 μL) were loaded with resin and equilibrated with IMAC equilibration buffer (50 mM buffer pH=6-8; 500 mM NaCl). Waste was removed using a positive pressure manifold. mRNA (~4,800 nt, >95% tail purity, >80% size purity) in the same buffer was added at a load of 15/L resin and incubated for 60 minutes. Flowthrough was collected using a positive pressure manifold. An IMAC elution buffer (50 mM Tris, pH=8; 200 mM imidazole) was added, incubated for 30 minutes, and the eluate was collected using a positive pressure manifold. RNA present in the flowthrough and eluate was quantified (by measuring absorbance at 260 nm) Results are shown in FIG. 5. The results show static binding of RNA to the Nuvia IMAC resin under slightly acidic conditions, e.g., pH<7, including pH 6.0.

Columns (5 mL) were prepared and loaded with mRNA (~4,800 nt, ~70% tail purity) in buffer (50 mM buffer, pH=6-8; 500 mM NaCl), with a retention time of 20 minutes, and flowthrough was collected. A chase was conducted using the same buffer and retention time of 20 minutes, and flowthrough was collected. Elution was conducted using an IMAC elution buffer (50 mM Tris, pH=8; 200 mM imidazole) and a retention time of 5 minutes, and the eluate was collected. Poly(A) RNA present in the flowthrough and eluate was quantified (by measuring absorbance at 260 nm). Results are shown in FIG. 6. The results show that, at pH<7.5, including at pH of from 6.0 to 7.0, pH does not appear to impact poly(A) selectivity.

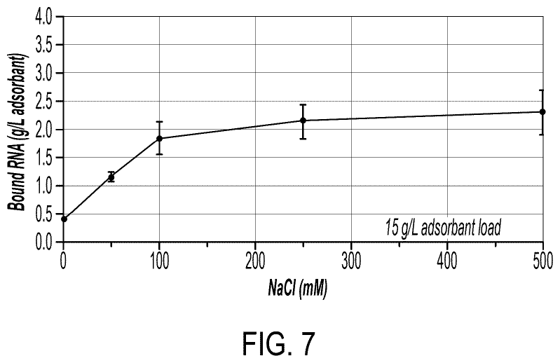

Microplates (50 μL) were loaded with resin and equilibrated with IMAC equilibration buffer (50 mM HEPES, pH=7; 0-500 mM NaCl). Waste was removed using a positive pressure manifold. mRNA (~4,800 nt, >95% tail purity, >80% size purity) formulated in the same buffer was added at a load of 15/L resin and incubated for 60 minutes. Flowthrough was collected using a positive pressure manifold. An IMAC elution buffer (50 mM Tris, pH=8; 200 mM imidazole) was added, incubated for 30 minutes, and the eluate was collected using a positive pressure manifold. RNA present in the flowthrough and eluate was quantified (by measuring absorbance at 260 nm) Results are shown in FIG. 7. The results show that static binding of RNA to the Nuvia IMAC resin depends on salt concentration, and that a salt concentration of, e.g., >100 mM NaCl is needed for effective binding.

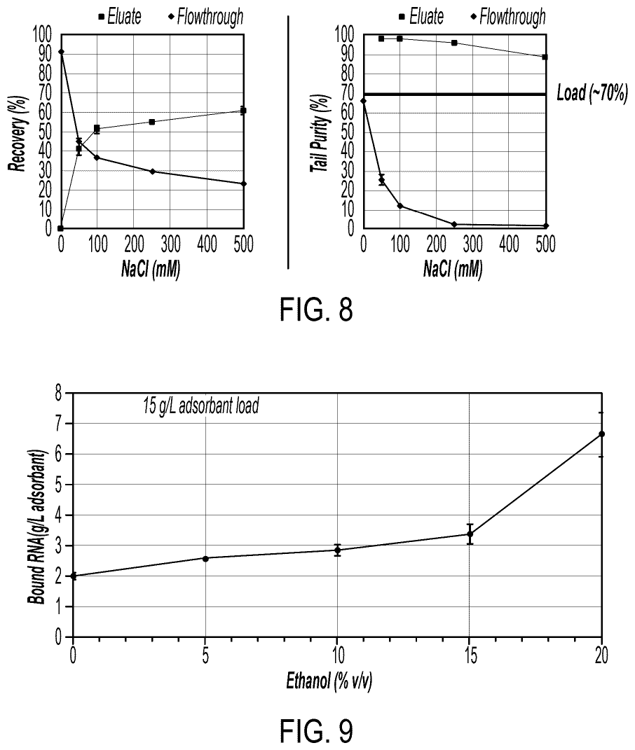

Columns (5 mL) were prepared and loaded with mRNA (~4,800 nt, ~70% tail purity) in buffer (50 mM HEPES, pH=7; 0-500 mM NaCl), with a retention time of 20 minutes, and flowthrough was collected. A chase was conducted using the same buffer and retention time of 20 minutes, and flowthrough was collected. Elution was conducted using an IMAC elution buffer (50 mM Tris, pH=8; 200 mM imidazole) and a retention time of 5 minutes, and the eluate was collected. Poly(A) RNA present in the flowthrough and eluate was quantified (by measuring absorbance at 260 nm). Results are shown in FIG. 8. The results show that increased salt concentration (e.g., >250 mM NaCl) improves RNA binding at the expense of selectivity for poly(A) RNA. For example, excess salt (>>25 mM, i.e., ~500 mM) appears to enhance nonspecific binding of tailless RNA, as indicated by the drop in tail purity.

Microplates (50 μL) were loaded with resin and equilibrated with IMAC equilibration buffer (50 mM HEPES, pH=7; 250 mM NaCl, 0-20% v/v ethanol). Waste was removed using a positive pressure manifold. mRNA (~4,800 nt, >95% tail purity, >80% size purity) formulated in the same buffer was added at a load of 15/L resin and incubated for 60 minutes. Flowthrough was collected using a positive pressure manifold. An IMAC elution buffer (50 mM Tris, pH=8; 200 mM imidazole) was added, incubated for 30 minutes, and the eluate was collected using a positive pressure manifold. RNA present in the flowthrough and eluate was quantified (by measuring absorbance at 260 nm) Results are shown in FIG. 9. The results show that ethanol enhances the RNA binding capacity of the Nuvia IMAC resin.

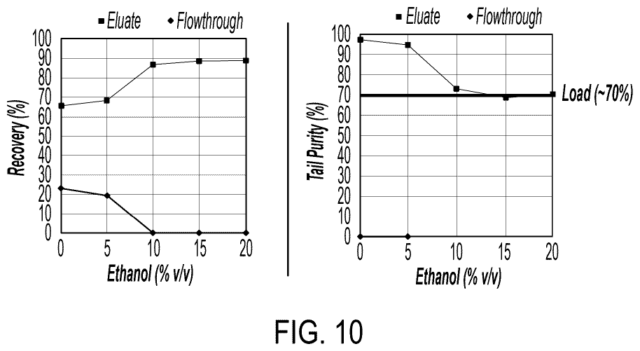

Columns (5 mL) were prepared and loaded with mRNA (~4,800 nt, ~70% tail purity) in buffer (50 mM HEPES, pH=7; 250 mM NaCl, 0-20% v/v ethanol), with a retention time of 20 minutes, and flowthrough was collected. A chase was conducted using the same buffer and retention time of 20 minutes, and flowthrough was collected. Elution was conducted using an IMAC elution buffer (50 mM Tris, pH=8; 200 mM imidazole) and a retention time of 5 minutes, and the eluate was collected. Poly(A) RNA present in the flowthrough and eluate was quantified (by measuring absorbance at 260 nm). Results are shown in FIG. 10. The results show that ethanol enhances nonspecific binding of tailless RNA to the Nuvia IMAC resin, as indicated by reduced tail purity at ethanol concentrations at or above 10% v/v.

Further studies were conducted to assess the ability to selectively wash off tailless RNA. It was found that an IMAC wash buffer of 50 mM HEPES, pH=7 and 0 or 250 mM NaCl was not effective to selectively wash off tailless RNA. (Data not shown).

### Example 3. IMAC Conditions for Selective Binding in Presence of His-Tagged Protein

The following IMAC conditions permit selective elution of poly(A) RNA (e.g., mRNA) in the presence of a His-tagged protein, such as His-tagged T7 RNA polymerase: pH 6 (load)>pH 8 (elution 1), 200 mM NaCl>imidazole (elution 2) With this protocol, the salt improves RNA recovery and may prevent His-tagged T7 polymerase (and other His-tagged proteins), from sequestering some of the RNA and preventing its elution.

Collectively, these results show that the Nuvia IMAC resin can be used to selectively isolate and purify poly(A) RNA (e.g., mRNA) under the conditions identified herein. The results surprisingly show that, under the conditions described herein the Nuvia IMAC resin can achieve a high tail purity (>95%), at least comparable to the oligo dT150 resin (~96%), which currently is the state of the art for RNA purification.

The Nuvia IMAC resin is capable of binding large (>4,000 nt) RNA molecules (possibly due to its large pore size, 900 Å), and exhibited the highest static binding capacity of all IMAC resins tested. As shown above, RNA capacity and poly(A) selectivity using the Nuvia IMAC is tunable (unlike the OligodT150 resin), such as by adjusting the IMAC conditions as described herein.

### Example 4. In Vitro Transcription (IVT) Compositions

As discussed above, the methods and apparatuses described herein are useful for purifying mRNA from an IVT composition. When mRNA is made via in vitro transcription (IVT), typically a construct comprising an open reading frame (ORF) of the gene of interest and other elements is transformed into a competent host cell, such as E. coli. Typically, the ORF is flanked by a 5′ untranslated region (UTR), which may contain a strong Kozak translational initiation signal and/or an alpha-globin 3′ UTR which may include an oligo (dT) sequence for templated addition of a poly-A tail. The ORF also may include various upstream or downstream additions (such as, but not limited to, β-globin, tags, etc.) and may contain multiple cloning sites which may have Xbal recognition.

A typical in vitro transcription reaction includes the following, where “Custom NTPs” is the input nucleotide triphosphate (NTP).

| 1 | Template cDNA | 1.0 | μg |
|---|---|---|---|
| 2 | 10x transcription buffer (400 mM | 2.0 | μl |
| | Tris-HCl pH 8.0, 190 mM MgCl₂, | | |
| | 50 mM DTT, 10 mM Spermidine) | | |
| 3 | Custom NTPs (25 mM each) | 7.2 | μl |
| 4 | RNase Inhibitor | 20 | U |
| 5 | T7 RNA polymerase | 3000 | U |
| 6 | dH₂0 | Up to | |
| | | 20.0 μL and | |
| 7 | Incubation at 37° C. for 3 hr-5 hrs. | | |

The crude IVT mix obtained after incubation may be stored at 4° C. overnight for cleanup the next day. 1 U of RNase-free DNase is then used to digest the original template (e.g., for 15 minutes at 37° C.). Thus, the resulting IVT composition may include linearized DNA plasmid, histidine-tagged T7 RNA polymerase, DNase (e.g., DNase I), nucleotide triphosphates, and buffer.

### Example 5. Isolation and Purification of mRNA Using IMAC

This example outlines a general method for isolating and purifying mRNA from an IVT composition using IMAC.

An IVT composition (e.g., prepared as described above) is used as starting material. If necessary, the pH of the composition is adjusted to a pH<7, e.g., by adding a suitable buffer, such as an MES buffer. NaCl is added to the composition to a concentration of 250 mM. Ethanol is added to a concentration of 15% v/v.

The composition is applied to an IMAC column. mRNA present in the IVT composition is retained on the column via binding between IMAC ligands and poly(A) tails of mRNA molecules. Other components of the IVT composition (i.e., other substances present in the composition), including non-histidine tagged protein, are collected in the flowthrough.

mRNA is eluted from the column using an elution buffer comprising 50 mM Tris at pH 8 and 200 mM NaCl.

If desired, histidine-tagged protein is eluted from the column using a Tris buffer comprising imidazole.

What is claimed is:

1. A method of purifying a polynucleotide having a poly(A) sequence, comprising:
contacting a composition comprising a polynucleotide having a poly(A) sequence with an immobilized metal affinity chromatography (IMAC) ligand, wherein the IMAC ligand binds the polynucleotide having a poly(A) sequence to form an IMAC-polynucleotide complex;
separating the composition from the IMAC-polynucleotide complex; and
recovering purified polynucleotide from the IMAC-polynucleotide complex,
wherein the polynucleotide comprises a poly(A) sequence having a length of about 20 adenine nucleotides or longer.

2. The method of claim 1, wherein the polynucleotide is a linear polynucleotide.

3. The method of claim 1, wherein the polynucleotide is a circular polynucleotide.

4. The method of any one of the preceding claims, wherein the polynucleotide is an mRNA.

5. The method of claim 4, wherein the poly(A) sequence is a poly(A) tail of the mRNA.

6. The method of any one of the preceding claims, wherein the IMAC ligand comprises an amine-carboxylic acid chelating group and a metal ion.

7. The method of claim 6, wherein the IMAC ligand comprises an amine-carboxylic acid chelating group selected from nitrilotriacetic acid (—CH(COOH)N(CH2COOH)₂) (NTA) and iminodiacetic acid (—N(CH₂COOH)₂ (IDA).

8. The method of any one of the preceding claims, wherein the IMAC ligand comprises a metal ion selected from Cu²⁺, Zn²⁺, Ni²⁺, and Co²⁺.

9. The method of any one of the preceding claims, wherein the IMAC ligand is immobilized on a substrate.

10. The method of claim 9, wherein the substrate comprises an IMAC matrix comprising a polymer having the IMAC ligand attached thereto.

11. The method of claim 10, wherein the IMAC matrix is a chromatography resin.

12. The method of any one of the preceding claims, wherein the contacting comprises passing the composition through a chromatography column containing the IMAC ligand.

13. The method of any one of the preceding claims, wherein the composition contacted with the IMAC ligand has a pH<7 and >5.

14. The method of any one of the preceding claims, wherein the composition contacted with the IMAC ligand has a pH of about 6.

15. The method of any one of the preceding claims, wherein the composition contacted with the IMAC ligand comprises NaCl at a concentration of from about 100 mM to about 250 mM.

16. The method of any one of the preceding claims, wherein the composition contacted with the IMAC ligand comprises ethanol at a concentration of about 10% v/v.

17. The method of any one of claims 1-15, wherein the composition contacted with the IMAC ligand does not include ethanol.

18. The method of any one of the preceding claims, wherein recovering purified polynucleotide from the IMAC-polynucleotide complex is effected using an elution buffer.

19. The method of claim 19, wherein the elution buffer comprises imidazole, has a pH of about 8, and comprises NaCl at a concentration of about 200 nM.

20. The method of any one of the preceding claims, wherein the polynucleotide has a length of from about 50 to about 5000 nucleotides.

21. The method of any one of the preceding claims, wherein the polynucleotide has a length of from about 3000 to about 5000 nucleotides.

22. The method of any one of the preceding claims, wherein the poly(A) sequence has a length of from about 50 to about 300 adenine nucleotides.

23. The method of any one of the preceding claims, wherein the composition comprises a histidine-tagged protein, and wherein the method is effective to purify the polynucleotide from the histidine-tagged protein.

24. The method of any one of the preceding claims, wherein the composition comprises tailless RNA, and wherein the method is effective to purify the polynucleotide from the tailless RNA.

25. The method of any one of the preceding claims, wherein the polynucleotide is an mRNA and the composition is an in vitro transcribed-(IVT) mRNA comprising the mRNA and histidine-tagged T7 RNA polymerase, wherein the method is effective to purify the mRNA from the T7 RNA polymerase.

26. The method of any one of the preceding claims, wherein the polynucleotide is an mRNA and the composition is an in vitro transcribed-(IVT) mRNA composition comprising the mRNA and linearized DNA plasmid, histidine-tagged T7 RNA polymerase, and nucleotide triphosphates.

27. The method of any one of the preceding claims, wherein the composition comprises a chelating agent, and the method further comprises, prior to contacting the composition with the IMAC ligand, conducting a buffer exchange to remove chelating agent from the composition.

28. The method of any one of the preceding claims, further comprising a heat denaturation step.

29. The method of any one of the preceding claims, wherein the composition does not contain DNA.

30. An immobilized metal affinity chromatography (IMAC) column comprising an IMAC-polynucleotide complex comprising an IMAC ligand bound to a polynucleotide having a poly(A) sequence, wherein the poly(A) sequence has a length of about 20 adenine nucleotides or longer.
